(19) United States
(12) Patent Application Publication
(10) Pub. No.: US 2021/0128638 A1
(43) Pub. Date: May 6, 2021

(54) CELLULAR, ORGAN, AND WHOLE-BODY REJUVENATION UTILIZING CORD BLOOD PLASMA AND PTEROSTILBENE

(71) Applicant: Therapeutic Solutions International, Inc., Oceanside, CA (US)

(72) Inventors: Thomas E. Ichim, Oceanside, CA (US); Timothy G. Dixon, Oceanside, CA (US)

(73) Assignee: Therapeutic Solutions International, Inc., Oceanside, CA (US)

(21) Appl. No.: 17/089,389

(22) Filed: Nov. 4, 2020

Related U.S. Application Data
(60) Provisional application No. 62/929,975, filed on Nov. 4, 2019.

(51) Int. Cl. A61K 35/51; A61K 31/09

(52) U.S. Cl. CPC A61K 35/51; A61K 2035/124; A61K 31/09

(57) ABSTRACT

Disclosed are methods, means, and protocols for stimulation of rejuvenation in single cells, organs, and organisms by administration of cord blood derived plasma, cord blood plasma concentrates, and cord blood derived exosomes together with pterostilbene. The invention describes the previously unexpected finding that addition of pterostilbene to cord blood enhances the rejuvenation properties of cord blood. Said rejuvenation properties include telomere preservation, reduction in beta galactosidase, and retention of cellular activities.

### CROSS-REFERENCE TO RELATED APPLICATIONS

This application claims the benefit of priority to U.S. Provisional Application No. 62/929,975, filed Nov. 4, 2019, the entire contents of which is incorporated herein by reference.

### FIELD OF THE INVENTION

The teachings herein are methods, means, and protocols for stimulation of rejuvenation in single cells, organs, and organisms.

### BACKGROUND OF THE INVENTION

Previous studies have demonstrated that connection of circulation between young animals and old animals results in a systemic anti-aging effect. For example, a study from Stanford University examined the influence of systemic factors on aged progenitor cells from hepatic tissues, by establishing parabiotic pairings (that is, a shared circulatory system) between young and old mice (heterochronic parabioses), thus exposing old mice to factors present in young serum. Notably, heterochronic parabiosis restored the activation of Notch signaling as well as the proliferation and regenerative capacity of aged satellite cells. The exposure of satellite cells from old mice to young serum enhanced the expression of the Notch ligand (Delta), increased Notch activation, and enhanced proliferation in vitro. Furthermore, heterochronic parabiosis increased aged hepatocyte proliferation and restored the cEBP-alpha complex to levels seen in young animals. These results suggest that the age-related decline of progenitor cell activity can be modulated by systemic factors that change with age [1]. In another study, a group from Harvard University found factors in young blood induce vascular remodeling, culminating in increased neurogenesis and improved olfactory discrimination in aging mice using similar heterochronic parabiosis models. Identification of molecular factors associated with improvement of cerebral vascularity and neurogenesis revealed a role for the molecule GDF11 [2].

Unfortunately, means of connecting circulation in mammals is extremely difficult and unethical. The current invention provides means of substitution a young mammal as a source of regenerative factors with an extracorporeal bioreactor containing regenerative cells.

### SUMMARY

Teachings herein are directed to methods of inhibiting senescence comprising: contacting an entity selected from the group consisting of: cell, organ, and organism, with a combination comprising a i) substance selected from the group consisting of: cord blood plasma, cord blood plasma concentrate, and cord blood plasma exosomes, and a ii) composition containing pterostilbene or an analogue thereof; in an amount sufficient to inhibit senescence in said cell, organ or organism.

Further teachings are directed to embodiments wherein said cord blood plasma is a non-cellular fraction of cord blood.

Further teachings are directed to embodiments wherein said cord blood plasma is autologous to the recipient.

Further teachings are directed to embodiments wherein said cord blood plasma is allogeneic to the recipient.

Further teachings are directed to embodiments wherein said cord blood plasma is xenogeneic to the recipient.

Further teachings are directed to embodiments wherein said cord blood plasma is administered together with pterostilbene.

Further teachings are directed to embodiments wherein said pterostilbene is administered at a concentration sufficient to activate NRF2.

Further teachings are directed to embodiments wherein said cord blood plasma is administered in the form of a concentrated solution, which is adjusted to physiological conditions.

Further teachings are directed to embodiments wherein said cord blood plasma is administered in the form of cord blood exosomes, wherein said exosomes express CD8.

Further teachings are directed to embodiments wherein said cord blood plasma is administered in the form of cord blood exosomes, wherein said exosomes express CD68.

### BRIEF DESCRIPTION OF THE DRAWINGS

FIG. 1 is a bar graph showing cord blood plasma suppression of senescence marker Beta Galactosidase is augmented by pterostilbene.

### DETAILED DESCRIPTION OF THE INVENTION

The invention teaches the previously unknown ability to pterostilbene to enhance rejuvenating activity of cord blood plasma.

In one embodiment, cord blood plasma is administered to cells, organs, or organisms in need of rejuvenating activity together with an appropriate concentration of pterostilbene sufficient to activate NRF2 more than 10% compared to baseline.

In some embodiments, cord blood plasma is concentrated by lyophilization or other means known in the art such as filtration, or chromatography, and subsequently administered together with pterostilbene at a concentration of pterostilbene sufficient to activate NRF2.

In another embodiment, cord blood plasma exosomes are administered with pterostilbene. Exosomes are purified and concentrated as described below. Indeed, the applicant has now demonstrated that membrane vesicles, particularly exosomes, could be purified, and possess ability to inhibit pain. In one embodiment, a strong or weak, preferably strong, anion exchange may be performed. In addition, in a specific embodiment, the chromatography is performed under pressure. Thus, more specifically, it may consist of high performance liquid chromatography (HPLC). Different types of supports may be used to perform the anion exchange chromatography. More preferably, these may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE®, POROS® SEPHAROSE®, SEPHADEX®, TRISACRYL®, TSK-GEL® SW OR PW®, SUPERDEX® TOYOPEARL® HW and SEPHACRYL®, for example, which are suitable for the application of this invention. Therefore, in a specific embodiment, this invention relates to a method of preparing membrane vesicles, particularly exosomes, from a biological sample such as a tissue culture containing fibroblasts, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalized.

In addition, to improve the chromatographic resolution, within the scope of the invention, it is preferable to use supports in bead form. Ideally, these beads have a homogeneous and calibrated diameter, with a sufficiently high porosity to enable the penetration of the objects under chromatography (i.e. the exosomes). In this way, given the diameter of exosomes (generally between 50 and 100 nm), to apply the invention, it is preferable to use high porosity gels, particularly between 10 nm and 5 .mu.m, more preferably between approximately 20 nm and approximately 2 .mu.m, even more preferably between about 100 nm and about 1 .mu.m. For the anion exchange chromatography, the support used must be functionalized using a group capable of interacting with an anionic molecule. Generally, this group is composed of an amine which may be ternary or quaternary, which defines a weak or strong anion exchanger, respectively.

Within the scope of this invention, it is particularly advantageous to use a strong anion exchanger. In this way, according to the invention, a chromatography support as described above, functionalized with quaternary amines, is used. Therefore, according to a more specific embodiment of the invention, the anion exchange chromatography is performed on a support functionalized with a quaternary amine. Even more preferably, this support should be selected from poly(styrene-divinylbenzene), acrylamide, agarose, dextran and silica, alone or in mixtures, and functionalized with a quaternary amine. Examples of supports functionalized with a quaternary amine include the gels SOURCEQ® MONO Q® Q SEPHAROSE.®., POROS® HQ and POROS® QE, FRACTOGEL® type gels and TOYOPEARL® SUPER Q® gels.

A particularly preferred support to perform the anion exchange chromatography comprises poly(styrene-divinylbenzene). An example of this type of gel which may be used within the scope of this invention is SOURCE Q® gel, particularly SOURCE 15 Q® (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 .mu.1 up to 10 ml or greater. In this way, the supports available have a capacity which may reach 25 mg of proteins/ml, for example. For this reason, a 100 .mu.1 column has a capacity of approximately 2.5 mg of proteins which, given the samples in question, allows the treatment of culture supernatants of approximately 21 (which, after concentration by a factor of 10 to 20, for example, represent volumes of 100 to 200 ml per preparation). It is understood that higher volumes may also be treated, by increasing the volume of the column, for example. In addition, to perform this invention, it is also possible to combine the anion exchange chromatography step with a gel permeation chromatography step. In this way, according to a specific embodiment of the invention, a gel permeation chromatography step is added to the anion exchange step, either before or after the anion exchange chromatography step. Preferably, in this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, are preferably used. As an illustration, for gel permeation chromatography, a support such as SUPERDEX® 200HR (Pharmacia), TSK G6000 (TosoHaas) or SEPHACRYL® S (Pharmacia) is preferably used. The process according to the invention may be applied to different biological samples. In particular, these may consist of a biological fluid from a subject (bone marrow, peripheral blood, etc.), a culture supernatant, a cell lysate, a pre-purified solution or any other composition comprising membrane vesicles.

In this respect, in a specific embodiment of the invention, the biological sample is a culture supernatant of membrane vesicle-producing fibroblast cells.

In addition, according to a preferred embodiment of the invention, the biological sample is treated, prior to the chromatography step, to be enriched with membrane vesicles (enrichment stage). In this way, in a specific embodiment, this invention relates to a method of preparing membrane vesicles from a biological sample, characterized in that it comprises at least: b) an enrichment step, to prepare a sample enriched with membrane vesicles, and c) a step during which the sample is treated by anion exchange chromatography and/or gel permeation chromatography.

In one embodiment, the biological sample is a culture supernatant treated so as to be enriched with membrane vesicles. In particular, the biological sample may be composed of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography. Therefore, a preferred method of preparing membrane vesicles according to this invention more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In another specific embodiment, the enrichment step comprises an affinity chromatography step, optionally preceded by a step of elimination of cells and/or cell debris (clarification). A preferred enrichment step according to this invention comprises (i) the elimination of cells and/or cell debris (clarification), (ii) a concentration and (iii) an affinity chromatography. The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 .mu.m, e.g. between 0.2 and 10 .mu.m, are preferentially used. It is particularly possible to use a succession of filters with a porosity of 10 .mu.m, 1 .mu.m, 0.5 .mu.m followed by 0.22 .mu.m.

A concentration step may also be performed, in order to reduce the volumes of sample to be treated during the chromatography stages. In this way, the concentration may be obtained by centrifugation of the sample at high speeds, e.g. between 10,000 and 100,000 g, to cause the sedimentation of the membrane vesicles. This may consist of a series of differential centrifugations, with the last centrifugation performed at approximately 70,000 g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to a preferred embodiment, the biological sample (e.g., the supernatant) is subjected to an ultrafiltration, preferably a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, GF, Sepracor).

Within the scope of the invention, the use of membranes with a cut-off threshold below 1000 kDa, preferably between 300 kDa and 1000 kDa, or even more preferably between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. Preferably, an affinity chromatography on a dye is used, allowing the elimination (i.e., the retention) of contaminants such as proteins and enzymes, for instance albumin, kinases, dehydrogenases, clotting factors, interferons, lipoproteins, or also co-factors, etc. More preferably, the support used for this chromatography step is a support as used for the ion exchange chromatography, functionalized with a dye. As specific example, the dye may be selected from Blue SEPHAROSE® Pharmacia), YELLOW 86, GREEN 5 and BROWN 10 (Sigma). The support is more preferably agarose. It should be understood that any other support and/or dye or reactive group allowing the retention (binding) of contaminants from the treated biological sample can be used in the instant invention.

In one embodiment a membrane vesicle preparation process within the scope of this invention comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample. In a preferred embodiment, step b) above comprises a filtration of the culture supernatant, followed by an ultrafiltration, preferably tangential. In another preferred embodiment, step b) above comprises a clarification of the culture supernatant, followed by an affinity chromatography on dye, preferably on Blue SEPHAROSE®.

In addition, after step c), the material harvested may, if applicable, be subjected to one or more additional treatment and/or filtration stages d), particularly for sterilisation purposes. For this filtration treatment stage, filters with a diameter less than or equal to 0.3 μm are preferentially used, or even more preferentially, less than or equal to 0.25 μm. Such filters have a diameter of 0.22 μm, for example. After step d), the material obtained is, for example, distributed into suitable devices such as bottles, tubes, bags, syringes, etc., in a suitable storage medium. The purified vesicles obtained in this way may be stored cold, frozen or used extemporaneously.

Therefore, a specific preparation process within the scope of the invention comprises at least the following steps: c) an anion exchange chromatography and/or gel permeation chromatography treatment of the biological sample, and d) a filtration step, particularly sterilizing filtration, of the material harvested after stage c). In a first variant, the process according to the invention comprises: c) an anion exchange chromatography treatment of the biological sample, and d) a filtration step, particularly sterilizing filtration, on the material harvested after step c).

In another variant, the process according to the invention comprises: c) a gel permeation chromatography treatment of the biological sample, and d) a filtration step, particularly sterilizing filtration, on the material harvested after step c). According to a third variant, the process according to the invention comprises: c) an anionic exchange treatment of the biological sample followed or preceded by gel permeation chromatography, and d) a filtration step, particularly sterilizing filtration, on the material harvested after step c).

In some embodiments, the composition containing an effective amount of pterostilbene comprises a pharmaceutically acceptable salt of pterostilbene. In some embodiments, the pterostilbene is isolated from a plant material. In some embodiments, the composition contains at least about a 0.75 wt. % pterostilbene component based on the dry weight of the plant isolate. In some embodiments, the pterostilbene is administered daily at a concentration ranging from about 0.007 to about 1500 mg pterostilbene component per kg metabolic weight. In some embodiments, pterostilbene is administered daily at a concentration of about 2.5 mg to about 10 mg of pterostilbene per kilogram of subject body weight. In some embodiments, pterostilbene is administered in capsules at a dose of about 200 mg at least twice daily. In some embodiments, pterostilbene is administered via an extract of Vaccinium berries, such as blueberries. In some embodiments, pterostilbene is administered with a combination of ingredients including, superoxide dismutase, curcumin, DMAE, alpha lipoic acid, and piperine.

In some embodiments, the pterostilbene is administered in the form of a sustained release (SR) formulation. In some embodiments, the SR formulation comprises a prodrug, or analog of N-pterostilbene, or a salt or solvate thereof. In some embodiments, the composition, upon oral administration, provides a therapeutically effective plasma concentration of pterostilbene over more than about 3 hours following the administration. In some embodiments, the pterostilbene formulation further comprises an immediate release (IR) component. In some embodiments, the IR component includes a prodrug, or analog of pterostilbene, or a salt or solvate thereof. In some embodiments, the composition, upon oral administration, provides a therapeutically effective plasma concentration of pterostilbene over about 45 minutes to about 20 hours following the administration. In some embodiments, the SR and/or IR component comprises a prodrug or analog of pterostilbene which is less polar than pterostilbene and possesses an increased absorbability profile in the lower gastrointestinal tract of a mammal. In some embodiments, the SR and/or IR component comprises a prodrug of pterostilbene selected from the group consisting of an ester prodrug, an amide prodrug, and an anhydride prodrug.

### EXAMPLE

### Cord Blood Plasma Suppression of Senescence Marker Beta Galactosidase is Augmented by Pterostilbene

Foreskin fibroblasts where obtained from ATCC and cultured according to manufacture instructions. Accelerated senescence was induced by exposure to indicated concentrations of H2O2 for 48 hours. Cells where cultured in control media (DMEM) or 10% cord blood plasma, or combination of cord blood plasma with 3 uMol Pterostilbene. Senescence was detected by fixing cells with 4% paraformaldehyde and SA-β-Gal was stained using senescent cells histochemical staining kit (Sigma Aldrich, St. Louis, Mo., USA). Three images per each well were collected, and the SA-β-Gal-stained cells were counted.

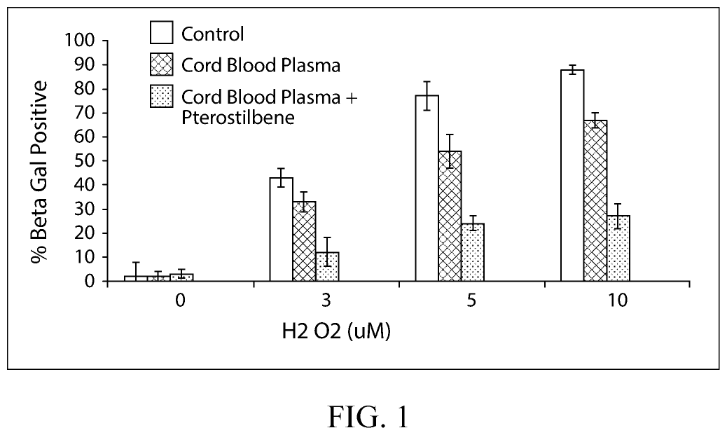

Results are shown in FIG. 1. In this assay pterostilbene alone did not exhibit a protective effect.

### REFERENCES

1. Conboy, I. M., et al., Rejuvenation of aged progenitor cells by exposure to a young systemic environment. Nature, 2005. 433(7027): p. 760-4.

2. Katsimpardi, L., et al., Vascular and neurogenic rejuvenation of the aging mouse brain by young systemic factors. Science, 2014. 344(6184): p. 630-4.

1. A method of inhibiting senescence comprising:
contacting an entity selected from the group consisting of: cell, organ, and organism,
with a combination comprising a
i) substance selected from the group consisting of: cord blood plasma, cord blood plasma concentrate, and cord blood plasma exosomes, and a
ii) composition containing pterostilbene or an analogue thereof;
in an amount sufficient to inhibit senescence in said cell, organ or organism.

2. The method of claim 1, wherein said cord blood plasma is a non-cellular fraction of cord blood.

3. The method of claim 1, wherein said cord blood plasma is autologous to the recipient.

4. The method of claim 1, wherein said cord blood plasma is allogeneic to the recipient.

5. The method of claim 1, wherein said cord blood plasma is xenogeneic to the recipient.

6. The method of claim 1, wherein said cord blood plasma is administered together with pterostilbene.

7. The method of claim 6, wherein said pterostilbene is administered at a concentration sufficient to activate NRF2.

8. The method of claim 1, wherein said cord blood plasma is administered in the form of a concentrated solution, which is adjusted to physiological conditions.

9. The method of claim 1, wherein said cord blood plasma is administered in the form of cord blood exosomes, wherein said exosomes express CD8.

10. The method of claim 1, wherein said cord blood plasma is administered in the form of cord blood exosomes, wherein said exosomes express CD68.
